(19) United States
(12) Patent Application Publication
(10) Pub. No.: US 2007/0202549 A1
(43) Pub. Date: Aug. 30, 2007

(54) Assessing risk of cerebrovascular thrombosis by measuring C4d on platelets

(75) Inventors: Joseph M. Ahearn, Sewickley, PA (US); Susan M. Manzi, Wexford, PA (US); Amy Kao, Allison Park, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: University of Pittsburgh, Pittsburgh, PA (US)

(21) Appl. No.: 11/521,643

(22) Filed: Sep. 14, 2006

Related U.S. Application Data
(60) Provisional application No. 60/717,525, filed on Sep. 14, 2005.

(51) Int. Cl. G01N 33/567; G06F 19/00

(52) U.S. Cl. 435/7.21; 702/21

(57) ABSTRACT

This invention provides for a convenient, economical, and sensitive test for assessing the risk of a person suffering from a thrombotic stroke. More specifically, it has been determined that persons at risk for cerebrovascular thrombosis have an elevated deposition of C4d on their platelets.

### RELATED APPLICATIONS

This application claims priority to U.S. provisional application No. 60/717,525, filed Sep. 14, 2005, the contents of which is incorporated herein by reference in the entirety.

### BACKGROUND OF THE INVENTION

After heart disease and cancers, stroke is the third leading cause of death in the developed countries. Each year in the United States, about 700,000 new or recurring strokes take place. Every 45 seconds, someone in this country suffers from a stroke. On average, we have a one-in-five chance to suffer from a stroke during our lifetime.

Besides the high prevalence, the result of a stroke can be devastating: about 30% of all strokes are fatal; 50-70% of survivors will have a mild disability; 15-30% of survivors will become severely disabled; and up to 20% of survivors will require institutional care for at least three month post-stroke. The cost of strokes is not just measured in the billions of dollars lost in work, hospitalization, and the care of survivors in nursing homes. The major cost or impact of a stroke is the loss of independence that occurs in 30% of the survivors. What was a self-sustaining and enjoyable lifestyle may lose most, if not all, of its quality after a stroke and other family members can find themselves in a new role as caregivers.

Although modern medicine provides advanced approaches for stroke diagnosis and treatment, there remains a need for new methods to rapidly, conveniently, and effectively identify individuals with high risk of stroke and to diagnose or monitor the condition in patients already with some symptoms of a stroke. The present invention meets this and other related needs.

### BRIEF SUMMARY OF THE INVENTION

In one aspect, this invention provides a method for assessing risk of cerebrovascular thrombosis in an individual. This method comprises the following steps: first, determining the platelet surface level of a complement pathway component C4d in the individual, and second, comparing the level of C4d of the individual with a standard control. In the event an increase in the level of C4d from the standard control is detected, an increased risk of cerebrovascular thrombosis is then indicated in this individual.

In some embodiments, the level of C4d is determined using an antibody that specifically binds C4d. Preferably, the C4d antibody is labeled, e.g., with a fluorescent moiety, which allows detection of the antibody by flow cytometry.

In other embodiments, the individual being tested has no clinical symptoms of cerebrovascular thrombosis; or the individual might have previously suffered from cerebrovascular thrombosis.

In another aspect, this invention provides a method for diagnosing or monitoring cerebrovascular thrombosis in an individual. This method comprises the following steps: first, determining the platelet surface level of a complement pathway component C4d in the individual, and second, comparing the level of C4d of the individual with a standard control. In the case an increase in the level of C4d from the standard control is detected, it is indicated that the individual is suffering from the condition of cerebrovascular thrombosis or a worsening of the condition.

In some embodiments, the level of C4d is determined using an antibody that specifically binds C4d. Preferably, the C4d antibody is labeled, e.g., with a fluorescent moiety, which allows detection of the antibody by flow cytometry.

In other embodiment, the individual being tested already has one or more clinical symptoms of cerebrovascular thrombosis.

In yet another aspect, this invention provides a computer readable medium for predicting, diagnosing, or monitoring cerebrovascular thrombosis in an individual. This computer readable medium comprises: (a) code for receiving data corresponding to a level of the complement pathway component C4d on the surface of platelets from an individual; (b) code for retrieving a standard control; and (c) code for comparing the data in (a) with the standard control in (b).

### BRIEF DESCRIPTION OF THE DRAWINGS

FIG. 1 illustrates the levels of C4d detected on platelet surface in 19 stroke patients and in normal healthy control subjects. The C4d levels are indicated by indirect immunofluorescence.

### DEFINITIONS

The term “cerebrovascular thrombosis,” as used herein, refers to a pathological process in which a blood clot builds up in a blood vessel in the brain and ultimately causes blockage of the blood vessel, which in turn leads to an ischemic stroke, a type of stroke characterized by damage to the brain cells due to insufficient blood flow and oxygen supply. In this application, this term encompasses any type of ischemic stroke, including the so-called transient ischemic attack (TIA). The clinical symptoms of cerebrovascular thrombosis include, but are not limited to, weakness or paralysis on one side of the body; a partial or complete loss of voluntary movement and/or sensation in a leg and/or arm; speech problems and weak muscles of the face, which can cause drooling; numbness or tingling in the leg, arm, or face; impaired balance, vision, and swallowing functions; difficulty breathing and even unconsciousness.

As used herein the “complement pathway” or “complement system” refers to a complex network of more than 30 functionally linked proteins that interact in a highly regulated manner to provide many of the effector functions of humoral immunity and inflammation, thereby serving as the major defense mechanism against bacterial and fungal infections. This system of proteins acts against invasion by foreign organisms via three distinct pathways: the classical pathway (in the presence of antibody), the alternative pathway (in the absence of antibody), and the lectin pathway. Once activated, the proteins within each pathway form a cascade involving sequential self-assembly into multimolecular complexes that perform various functions intended to eradicate the foreign antigens that initiated the response. For a review of the complement pathway, see, e.g., Sim and Tsiftsoglou, Biochem. Soc. Trans. 32:21-27 (2004).

The classical pathway is usually triggered by an antibody bound to a foreign particle. It consists of several components that are specific to the classical pathway and designated C1, C4, C2. Sequentially, binding of C1q to an antigen-antibody complex results in activation of C1r and C1s (both are serine proteases), and activated C1s cleaves C4 and C2 into, respectively, C4a and C4b and C2a and C2b. Fragments C4b and C2a assemble to form C4b2a, which cleaves protein C3 into C3a and C3b, which completes activation of the classical pathway. Fragments C4b and C3b are subject to further degradation by Factor I. This factor cleaves C4b to generate C4d and also cleaves C3b, to generate iC3b followed by C3d. Thus, activation of the classical pathway of complement can lead to deposition of a number of fragments, such as C4d, iC3b, and C3d, on immune complexes or other target surfaces. Such targets include cells circulating in the blood, e.g., lymphocytes and other white blood cells, erythrocytes, and platelets.

Components of the complement pathway include proteins C1, C4, C2, C3, and fragments thereof, e.g., C1q, C1r, C1s, C4a, C4b, C2a, C2b, C4b2a, C3a, C3b, C4c, C4d, iC3b, C3d, C31, C3dg. Also included are C5, C5b, C6, C7, C8, C9, C1inh, MASP2, CR1, DAF, MCP, CD59, C3aR, C5aR, C1qR, CR2, CR3, and CR4, as well as other complement pathway components, receptors and ligands not listed specifically herein.

As used herein, “the platelet surface level of a complement pathway component C4d” refers to the amount of C4d found on the surface of a predetermined number of platelets obtained from an individual person.

A “standard control” as used herein refers to a platelet surface C4d level used as a comparison basis in practicing a method of the present invention. Such a standard control should reasonably indicate the level of platelet surface C4d in an average individual who is not suffering from or at risk of developing cerebrovascular thrombosis, and is not suffering from or at risk of any other diseases or conditions that tend to elevate platelet surface C4d level. Preferably, a standard control reflects the platelet surface C4d level from a healthy individual with medical background, as well as in age, gender, ethnicity, etc., comparable to the individual whose platelet surface C4d level is being tested.

“An increase in the level of C4d from the standard control” as used herein refers to a positive change in value from the standard control. Such an increase is usually at least 50%. The increased level of C4d is preferably at least 2-fold, more preferably at least 5-fold, and most preferably at least 10-fold of the control level.

An “antibody” refers to a glycoprotein of the immunoglobulin family or a polypeptide comprising fragments of an immunoglobulin that is capable of noncovalently, reversibly, and in a specific manner binding a corresponding antigen. The typical antibody structural unit is a tetramer. Each tetramer is composed of two identical pairs of polypeptide chains, each pair having one “light” (about 25 kD) and one “heavy” chain (about 50-70 kD), connected through a disulfide bond. The recognized immunoglobulin genes include the κ, λ, α, γ, δ, ε, and μ constant region genes, as well as the myriad immunoglobulin variable region genes. Light chains are classified as either κ or λ. Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (V_{L}) and variable heavy chain (V_{H}) refer to these regions of light and heavy chains respectively.

The term antibody, as used herein, includes both monoclonal and polyclonal antibodies, and encompasses antibodies raised in vivo, e.g., produced by an animal upon immunization by an antigen, and antibodies generated in vitro, e.g., generated by hybridomas. As used in this application, antibodies that specifically recognize the same antigen, e.g., a pathogenic organism, are regarded as “one antibody,” regardless of whether they actually bind to the same or to separate antigenic epitopes of the antigen.

For preparation of monoclonal or polyclonal antibodies, any technique known in the art can be used (see, e.g., Kohler & Milstein, Nature 256:495-497, 1975; Kozbor et al., Immunology Today 4:72, 1983; Cole et al., Monoclonal Antibodies and Cancer Therapy, pp. 77-96. Alan R. Liss, Inc., 1985). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., supra; Marks et al., Biotechnology, 10:779-783, 1992).

The term “specific binding” or “specifically binds,” when used to describe the binding reaction between an antibody to a protein (e.g., C4d), refers to the characteristic of the binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to a component of the complement pathway or to a surface marker of platelets, polymorphic variants, alleles, orthologs, and conservatively modified variants, or splice variants, or portions thereof, can be selected to obtain only those polyclonal antibodies that are specifically immunoreactive with the component of the complement pathway or the platelet surface marker and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

### DETAILED DESCRIPTION OF THE INVENTION

I. Introduction

The present invention provides a novel method for expediently assessing a person's risk for a future episode of cerebrovascular thrombosis, a type of stroke caused by the blockage of a blood vessel in the brain. According to this method, a patient's blood sample is taken and the level of platelet surface C4d, a component of the complement system, is measured. This C4d level is then compared with an established standard control, which reflects the average amount of platelet surface C4d found in a healthy person not at risk of cerebrovascular thrombosis or other conditions that tend to elevate C4d level on the platelets. An increase in the C4d level indicates a heighten risk for an individual to suffer from cerebrovascular thrombosis. Similarly, the platelet surface C4d levels can also be used to diagnose cerebrovascular thrombosis or monitor the condition of cerebrovascular thrombosis in a patient, where a higher-than-normal C4d level indicates that presence of cerebrovascular thrombosis or a worsening of the condition in a patient.

II. Determination of Platelet Surface C4d Level

The present invention involves conducting assays on platelets obtained from an individual to determine the level of C4d, a complement pathway component, deposited on the surface of platelets. The C4d level of is then used to predict, diagnose, or monitor cerebrovascular thrombosis in the individual.

The procedure of determining platelet surface C4d level begins with acquisition of a blood sample from a patient. Generally, patient blood samples are treated with EDTA (ethylenediaminetetraacetate) to inhibit complement activation, and can be maintained at room temperature or under cold conditions. Assays are run preferably within 24 hours from sample collection.

Many methods for isolating platelets are known in the art. For instance, density gradient centrifugation methods are frequently used, where palates are separated from other components of the blood based on difference in density following one or more rounds of centrifugation process. Isolation of platelets can also be achieved by an affinity-based method. For example, an antibody specific to a platelet surface marker (e.g., CD42b) can be first attached to a solid support (such as a Sepharose column), then contacted with a blood sample under conditions that permits the formation of antigen-antibody complex. While other undesired blood components are washed away, platelets can be recovered from the solid support.

In some embodiments of the present invention, fluorescence activated cell sorting, or FACS, is used to determine the number of platelets while other blood components are present in a sample. FACS is a technique used to separate cells according to their surface content of particular molecules of interest. A molecule of interest can be specific for a type of cell or for particular cell state. The molecule of interest can be fluorescently labeled directly by binding to a fluorescent dye, or by binding to a second molecule, which has been fluorescently labeled, e.g., an antibody, lectin, or aptamer that has been fluorescently labeled and that specifically binds to the molecule of interest. Thus, platelet specific markers can by used to distinguish platelets from other components of the blood such as red or white blood cells in a blood sample.

Isolation of platelets also refers to gating techniques used to assay platelets during flow cytometric analysis. A labeled marker specific for platelets (e.g., a labeled anti-CD42b monoclonal antibody) is used to determine the amount of platelets in a sample. A second labeled marker (e.g., a labeled anti-C4d antibody) is then used to determine the level of C4d on the surface of the platelets.

In one embodiment of the present invention, platelet surface C4d level is determined to predict, diagnose, or monitor the progression of cerebrovascular thrombosis in individuals. The platelets are isolated or detected using platelet-specific antibodies e.g., anti-CD42b antibodies. In some embodiments, determination of the level of platelet surface C4d may be achieved by a number of methods including flow cytometry, ELISA using purified platelet preparations, and radioimmunoassay. In one embodiment of this invention, the determination of the platelet surface C4d level is made using flow cytometric methods, with measurements taken by direct or indirect immunofluorescence using polyclonal or monoclonal antibodies specific for C4d. The mean fluorescence channel (MFC) for the platelet surface C4d can be determined. Detection and quantification of C4d on the surface of platelets is described in, e.g., WO 04/093647 and U.S. Ser. No. 60/463,447, both of which are herein incorporated in their entirety by reference for all purposes.

III. Kits

Kits for conducting the assays for predicting, diagnosing, or monitoring of cerebrovascular thrombosis in a patient are a part of this invention. The kits may comprise any of the various reagents needed to perform the methods described herein. For example, a kit adapted for the immunofluorescence assays generally comprises a conjugate of a monoclonal antibody specific for C4d with a first fluorescent moiety, and preferably also a conjugate of a monoclonal antibody specific for a platelet surface marker (e.g., CD42b) with a second, different fluorescent moiety. Additionally, the kits can comprise instructional material for the user and such other material as may be needed in carrying out assays of this type, for example, buffers, radiolabelled antibodies, colorimeter reagents, etc.

The antibodies for use in these methods and kits are known in the art and available commercially. For example, monoclonal antibodies specific for CD42b are available from commercial suppliers such as BIODESIGN International (Saco, Me.) and Yorkshire Bioscience (United Kingdom). Anti-C4d antibodies are available from Quidel Corp. (San Diego, Calif.) and are generally described in Rogers, J., N. Cooper, et al. PNAS 89:10016-10020 (1992); Schwab, C. et al. Brain. Res. 707(2):196 (1996); Gemmell, C. J. Biomed. Mater. Res. 37:474-480 (1997); and, Stoltzner, S. E., et al. Am. J. Path. 156:489-499 (2000).

IV. Diagnosis and Monitoring

A. Diagnosis

Diagnosis of a patient with cerebrovascular thrombosis or with an increased risk of developing cerebrovascular thrombosis is carried out by comparing the level of platelet surface C4d in this patient with a base value or standard control for the quantity of C4d that is typically present on the surface platelets in normal individuals. In normal individuals, the level of C4d on the surface of platelets is very low to not detectable. When using flow cytometric measurement with indirect immunofluorescence, the MFC of C4d on platelet surface of healthy individuals ranged from −1.17 to 0.87 (mean −0.39). In contrast, the MFC of platelet surface C4d in two patients suffering from cerebrovascular thrombosis was observed to be 6.00 and 16.53.

B. Monitoring of Patients

A particular feature of the methods of this invention is to indicate or reflect the progress of cerebrovascular thrombosis that has occurred in a patient during the preceding several weeks or even several months. It is possible, using the claimed methods, to identify the worsening of cerebrovascular thrombosis that has previously occurred, or to predict a subsequent occurrence of cerebrovascular thrombosis based on the persistence of elevated level of C4d deposited on the surface of platelets.

V. Automation and Computer Software

The determination of platelet surface C4d level, and the diagnostic and monitoring methods described above can be carried out manually, but often are conveniently carried out using an automated system and/or equipment, in which the blood sample is analyzed automatically to make the necessary determination or determinations, and the comparison with a standard control or reference value is performed automatically, using computer software appropriate to that purpose.

Thus, in one aspect, the invention comprises a method for predicting, diagnosing, or monitoring cerebrovascular thrombosis in an individual comprising (a) automatically determining, in a blood sample from the individual containing platelets, the level of C4d deposited on surfaces of platelets in the sample, and (b) automatically comparing the platelet surface C4d level with a standard control or reference value that reflects the average C4d level found on a normal, healthy individual's platelets.

In another aspect, the invention comprises a method for predicting, diagnosing, or monitoring cerebrovascular thrombosis in an individual comprising (a) automatically determining, in a blood sample from the individual containing platelets, the level of C4d deposited on surface of platelets in the sample, and (b) automatically comparing C4d level with a standard control indicating the average C4d level found on a normal, healthy individual's platelets.

Computer software, or computer-readable media for use in the methods of this invention includes a computer readable medium, which comprises:

(1) code for receiving data corresponding to a level of C4d deposited on the surface platelets in a blood sample;

(2) code for retrieving a standard control, which indicate the average C4d level found on a normal, healthy individual's platelets; and

(3) code for comparing the data in (a) with the standard control of (b).

In some embodiments of the invention, more than one standard controls may be stored in a memory associated with a digital computer. After data corresponding to the level of platelet surface C4d are obtained (e.g., from an appropriate analytical instrument), the digital computer may compare the C4d level with one or more appropriate standard controls. After this comparison takes place, the digital computer can automatically determine if the data corresponding to the C4d level is associated with cerebrovascular thrombosis.

In some embodiments of the invention, more than one C4d levels may be stored in a memory associated with a digital computer. For instance, the platelet surface C4d level from a particular individual may be measured at different points in time for the purpose of monitoring the progress of cerebrovascular thrombosis. After new data corresponding to C4d levels are obtained (e.g., from an appropriate analytical instrument), the digital computer can compare the C4d levels with the appropriate standard control(s), and/or with the platelet C4d levels recorded at previous time points. After this comparison takes place, the digital computer can automatically determine if the data corresponding to the C4d levels indicate an improvement or deterioration of cerebrovascular thrombosis in the patient.

Accordingly, one aspect of the invention may be embodied by computer code that is executed by a digital computer. The digital computer may be a micro, mini, or large frame computer using any standard or specialized operating system such as a Windows™ based operating system. The code may be stored on any suitable computer readable media. Examples of computer readable media include magnetic, electronic, or optical disks, tapes, sticks, chips, etc. The code may also be written by those of ordinary skill in the art and in any suitable computer programming language including, C, C++, etc.

### EXAMPLES

The following examples are provided by way of illustration only and not by way of limitation. Those of skill in the art will readily recognize a variety of non-critical parameters that could be changed or modified to yield essentially the same or similar results.

### Example 1

### Measuring Platelet Surface C4d Level in Healthy Individuals—Establishing Standard Controls

Twenty-five healthy individuals were studied. As shown in Table I, C4d was not detected on the surface of platelets in each of the twenty-five healthy individuals. Samples of 1 mL of EDTA-anticoagulated peripheral blood were taken from each individual and used as a source of platelets. The platelets were washed and resuspended in FACS buffer. Levels of C4d and CD42b were measured by two color indirect immunofluorescence using monoclonal antibodies specific for C4d and CD42b, respectively, Levels of C4d and CD42b were quantified by flow cytometry using a FACSCalibur cytometer (Becton Dickinson, Franklin Lakes, N.J.). The platelets were identified by forward and side scatter and CD42b-fluorescence, and the mean fluorescence channel (MFC) was determined for C4d as well as for CD42b.

TABLE I
Healthy Controls (n = 25)
Mean MFC = −0.39
Range (−1.17) to (+0.87)

| Platelet C4d | MFC |
|---|---|
| 2003 | −0.28 |
| 2005 | −0.23 |
| 2006 | −0.51 |
| 2007 | −0.05 |
| 2008 | 0.20 |
| 2009 | 0.15 |
| 2010 | −0.39 |
| 2011 | −0.71 |
| 2013 | −0.96 |
| 2017 | 0.87 |
| 2020 | −0.29 |
| 2021 | −0.56 |
| 2022 | 0.38 |
| 2025 | −0.73 |
| 2026 | −0.24 |
| 2027 | −0.34 |
| 2028 | −0.74 |
| 2029 | −0.05 |
| 2030 | −0.51 |
| 2031 | −1.03 |
| 2032 | −0.42 |
| 2034 | −0.71 |
| 2035 | −0.86 |
| 2036 | −0.48 |
| 2037 | −1.17 |

particularly, blood was drawn into 4 cc Vacutainer tubes containing 7.2 mg EDTA as an anticoagulant (Becton Dickinson), and processed within two hours. Whole blood was diluted 1/10 in phosphate buffered saline (PBS). 10 μl aliquots of the diluted blood were immufluorescently labeled for flow cytometry with 0.25 μg of PE-labeled anti-CD 42b monoclonal antibody (Becton Dickinson Immunocytometry Systems, San Jose, Calif.) to identify platelets, and 0.25 μg of anti-C4d monoclonal antibodies (Quidel Corp., San Diego, Calif.) conjugated to Alexa Fluor 488 (Molecular Probes, Eugene, Oreg.) or the isotype control MOPC21 (obtained from American Type Culture Collection, Manassas, Va.). Samples were incubated 10 min at room temperature, then diluted with 0.5 ml cold PBS and analyzed on a FACSCalibur flow cytometer (Becton Dickinson Immunocytometry Systems, San Jose, Calif.). Platelets were electronically gated by forward scatter properties and expression of CD42b, a platelet-specific marker. Nonspecific binding of immunoglobulins to platelets was determined by performing identical assays in parallel using the isotype control antibody MOPC21. Specific binding of anti-C4d and anti-CD42b were determined by subtracting the MFC obtained with MOPC21 from the MFC obtained with anti-C4d and anti-CD 42b, respectively.

### Example 2

### Increased Platelet C4d Levels in Cerebrovascular Thrombosis Patients

This example describes assays detecting higher-than-normal platelet surface C4d levels in patients suffering from cerebrovascular thrombosis.

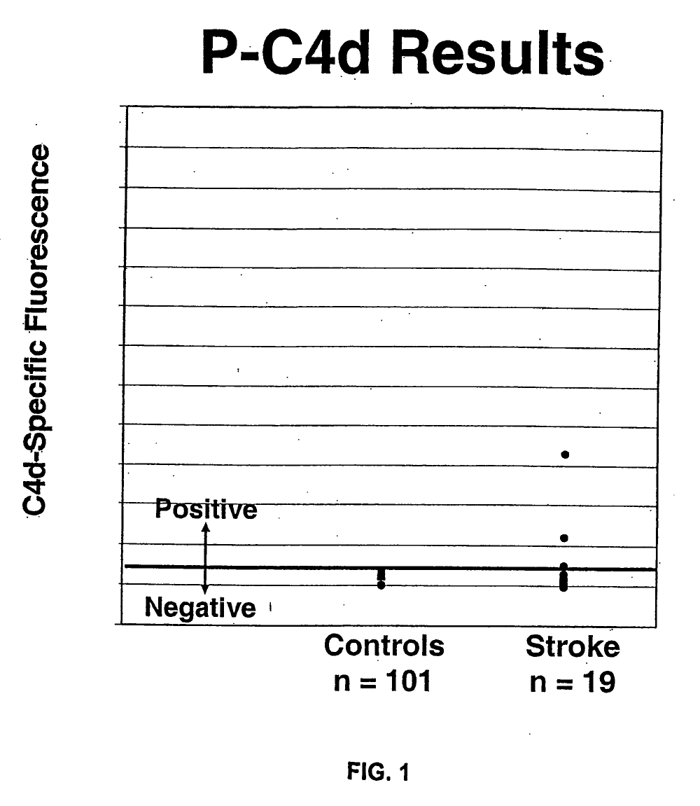

19 patients aged from 40-60 with acute thrombotic stroke were recruited in the study. The patients' platelet C4d levels were determined using methods previously described (see, e.g., WO 04/093647) and compared to a standard control established above. Two male patients, age 51 and 54, showed significantly elevated platelet C4d levels. See FIG. 1. This result indicates platelet surface C4d level is an indicator of risk for premature cerebrovascular thrombosis in an individual and can serve as a biomarker for diagnosing cerebrovascular thrombosis or monitoring response to stroke therapy in patients already demonstrating one or more symptoms of a thrombotic stroke.

### Example 3

### Platelet C4d Levels Correlate with Stroke Severity

48 patients suffering from acute ischemic stroke were included in this study. These patients were of a relatively young age (median age 51 years with a standard deviation of 12.8 years). 12.5% of the patients presented a platelet C4d level elevated from a standard control that was established according to the method described in Example 1. Furthermore, acute ischemic stroke patients with platelet C4d elevated from the standard control had significantly greater stroke severity as determined by the NIH Stroke Scale than those with platelet C4d within the levels of standard control (median: 16 versus 3 respectively; p=0.009).

All patents, patent applications, and other publications cited in this application, including published amino acid or polynucleotide sequences, are incorporated by reference in the entirety for all purposes.

What is claimed is:

1. A method for assessing risk of cerebrovascular thrombosis in an individual comprising the steps of:
(a) determining the platelet surface level of a complement pathway component C4d in the individual, and
(b) comparing the level of C4d of the individual with a standard control,
wherein an increase in the level of C4d from the standard control indicates an increased risk of cerebrovascular thrombosis in the individual.

2. The method of claim 1, wherein the level of C4d is determined using an antibody that specifically binds C4d.

3. The method of claim 1, wherein the C4d antibody is labeled.

4. The method of claim 3, wherein the C4d antibody is labeled with a fluorescent moiety.

5. The method of claim 3, wherein the C4d antibody is detected by flow cytometry.

6. The method of claim 1, wherein the individual has no clinical symptoms of cerebrovascular thrombosis.

7. The method of claim 1, wherein the individual has previously suffered from cerebrovascular thrombosis.

8. A method for diagnosing or monitoring cerebrovascular thrombosis in an individual comprising the steps of:
(a) determining the platelet surface level of a complement pathway component C4d in the individual, and
(b) comparing the level of C4d of the individual with a standard control,
wherein an increase in the level of C4d from the standard control indicates the presence of cerebrovascular thrombosis or a worsening of cerebrovascular thrombosis in the individual.

9. The method of claim 8, wherein the level of C4d is determined using an antibody that specifically binds C4d.

10. The method of claim 8, wherein the C4d antibody is labeled.

11. The method of claim 10, wherein the C4d antibody is labeled with a fluorescent moiety.

12. The method of claim 10, wherein the C4d antibody is detected by flow cytometry.

13. The method of claim 8, wherein the individual has one or more clinical symptoms of cerebrovascular thrombosis.

14. A computer readable medium for predicting, diagnosing, or monitoring cerebrovascular thrombosis in an individual, the computer readable medium comprising:
(a) code for receiving data corresponding to a level of the complement pathway component C4d on the surface of platelets from an individual;
(b) code for retrieving a standard control; and
(c) code for comparing the data in (a) with the standard control in (b).
